(19) United States
(12) Patent Application Publication
(10) Pub. No.: US 2006/0089593 A1
(43) Pub. Date: Apr. 27, 2006

(54) Needle-free injection device for individual users

(76) Inventors: Sergio Landau, Laguna Niguel, CA (US); Christopher Sautter, Portland, OR (US); John L. Staylor, Wilsonville, OR (US)

Correspondence Address: KOLISCH HARTWELL, P.C., 200 PACIFIC BUILDING, 520 SW YAMHILL STREET, PORTLAND, OR, 97204, US

(21) Appl. No.: 11/069,538

(22) Filed: Feb. 28, 2005

Related U.S. Application Data
(63) Continuation-in-part of application No. 10/976,342, filed on Oct. 26, 2004.

(51) Int. Cl. A61M 5/30; A61M 5/00; A61M 5/20; A61M 5/315

(52) U.S. Cl. 604/68; 604/110; 604/135; 604/234; 604/220

(57) ABSTRACT

A needle-free injector for injecting fluid from an injection cartridge that has a longitudinal axis, an injection orifice at a distal end and a displaceable plunger. The needle-free injector includes an injector body having a longitudinal axis and a side opening to receive the cartridge from a position laterally offset from the injector body longitudinal axis. The needle-free injector further includes a system disposed in the injector body for providing injection power and a ram for transferring power from the system to the plunger. The ram is disposed in the injector body and is axially aligned with the longitudinal axis of the cartridge when the cartridge is in position in the injector body. The needle-free injector further includes a closure mechanism for closing the side opening of the injector body after the cartridge is in position in the injector body to lock the cartridge in place in the injector body. The disclosure also includes other apparatus and methods as discussed in detail herein.

This application is also a continuation-in-part of U.S. patent application Ser. No. 10/976,342 filed Oct. 26, 2004 and also claims the benefit of U.S. provisional patent application No. ______ filed Feb. 15, 2005 under Express Mail No. EV493217799US entitled Needle-Free Injection Device for Individual Users, which is hereby incorporated by reference in its entirety for all purposes.

### BACKGROUND

Needle-free injection systems provide an alternative to standard fluid delivery systems, which typically uses a needle adapted to penetrate the outer surface of an injection site. Typically, needle-free injection systems are designed to eject the fluid from a fluid chamber with sufficient pressure to allow the fluid to penetrate the target to the desired degree. For example, common applications for needle-free injection systems include delivering intradermal, subcutaneous and intramuscular injections into or through a recipient's skin. For each of these applications, the fluid must be ejected from the system with sufficient pressure to allow the fluid to penetrate the tough exterior dermal layers of the recipient's skin.

When using the same device to deliver inoculations, immunizations or the like, to different individuals, preventing cross-contamination between injection recipients and prevention of contamination of the filling source must be a priority. Thus, it is desirable to provide a device that allows a user to move with reasonable speed from one injection recipient to another while maintaining adequate protections against cross-contamination. In addition, it will often be desirable to obtain the above advantages while also keeping waste to a minimum (e.g., by avoiding unnecessary disposal of portions of the injection system).

It is also desirable in many applications that an injector be relatively small, hand-held, and ergonomically comfortable so that it can be easily handled by the health care provider. When a spring loaded injector is being used, it is also desirable that the injector spring be easily compressed. These and other advantages of the preferred embodiments will be apparent as this description continues.

### SUMMARY

A needle-free injector is provided for injecting fluid from an injection cartridge that has a longitudinal axis, an injection orifice at a distal end and a displaceable plunger. The injector also includes an injector body having a longitudinal axis and a side opening to receive the cartridge from a position laterally offset from the injector body longitudinal axis. A closure mechanism is included for closing the side opening of the injector body after the cartridge is in position in the injector body with the longitudinal axes of the cartridge and the injector body coincident in order to lock the cartridge in place in the injector body.

Another aspect of the invention is a method for loading an injection cartridge into a needle-free injector, including the following steps: selecting an injection cartridge that has a longitudinal axis; selecting an injector body that has a longitudinal axis, a side opening and a closure mechanism for selectively closing the side opening; opening the closure mechanism of the injector body; moving the injector cartridge laterally from a position that is laterally offset from the longitudinal axis of the injector body through the side opening of the injector body into position in the injector body with the longitudinal axes of the injector body and the cartridge in alignment; and closing the side opening of the injector body to lock the cartridge in place in the injector body.

The invention alternatively provides a spring-loaded needle-free injector having an injector body, a main spring, and a winder that is rotatable in either the first direction or a second direction, and which can compress the main spring by rotating in the first direction. Also included is a ratchet mechanism in the form of a first and a second toothed member with at least one of the members being spring biased toward the other member, the ratchet mechanism being interconnected with the winder so that winding the winder in the first direction causes both toothed members to rotate, but winding the winder in the second direction causes rotation of only the first toothed member.

Yet another aspect of the invention is a method for compressing the spring of a spring-loaded needle-free injector which includes the steps of selecting an injector body having a main spring, a winder that is rotatable in either a first direction or a second direction and which compresses the main spring by rotating in the first direction, and a ratchet mechanism having rotatable first and second members with sloped teeth. At least one of the members is spring biased toward the other member. The ratchet mechanism is interconnected with the winder so that winding the winder in the first direction causes both toothed members to rotate but winding the winder in the second direction causes rotation of only the first toothed member. The winder is wound in the first direction, causing both of the members to rotate to compress the main spring, and then is wound in the second direction, causing only one of the members to rotate. The winder is then wound again in the first direction, causing both of the members to rotate to further compress the main spring.

Another aspect of the invention is a spring-loaded needle-free injector having a main body with distal and proximal ends and a longitudinal axis. An injection cartridge is provided that is mounted to the distal end of the main body, the cartridge having a fluid chamber defined therein and an injection orifice at the distal end. A trigger sleeve is slidably mounted to the main body, and a trigger lock that prevents the trigger from sliding with respect to the main body when locked is also provided. A firing mechanism that is actuated by unlocking the trigger lock and then sliding the trigger sleeve toward the distal end of the main body is also provided to fire the injector.

Yet another aspect of the invention is a method of injecting fluid from a needle-free injector that includes the following steps: selecting an injector having a main body with distal and proximal ends; slidably mounting a trigger sleeve to the main body, the trigger sleeve having the capability of actuating a firing mechanism when slid along the main body in a distal direction, the trigger sleeve having a trigger lock that can be locked and unlocked and which prevents the trigger sleeve from sliding with respect to the main body when the trigger lock is locked; mounting an injection cartridge adjacent the distal end of the main body, the cartridge including a fluid chamber defined therein and an injection orifice at the distal end thereof; and unlocking the trigger lock and sliding the trigger sleeve toward the distal end of the main body to fire the injector and cause the fluid to be ejected out of the fluid chamber and through the injection orifice.

The invention could also provide a needle free injector which includes an injection cartridge with a plunger and an injection orifice, an injector body, a system for providing injection power, a ram disposed in the injector body for transferring injection power from the system to the plunger, and a frangible member mounting the ram to the plunger such that the ram and plunger can be retracted under one amount of force to fill the cartridge with fluid, and such that when the ram and plunger are driven forward under another amount of force that is greater than the one amount of force, the ram breaks the frangible member but still allows the ram and plunger to drive fluid from the cartridge through the injection orifice. Therefore, in the event someone attempts to retract the ram and plunger to re-fill the cartridge, the ram will retract but the plunger will not because the frangible member has been broken.

A method for preventing the re-loading of a cartridge is also provided that includes the steps of selecting an injection cartridge having a plunger and an injection orifice, and selecting an injector body in which the cartridge can be removably mounted and having a system for providing injection power, a ram for transferring injection power from the system to the plunger, and a frangible member mounting the ram to the plunger such that the ram and plunger can be retracted under one amount of force to fill the cartridge with fluid, and when the ram and plunger are driven forward under another amount of force that is greater than the one amount of force, the ram breaks the frangible member but still allows the ram and plunger to drive fluid from the cartridge through the injection orifice. Therefore in the event someone attempts to retract the ram and plunger to re-fill the cartridge, the ram will retract but the plunger will not because the frangible member has been broken.

Yet another aspect of the invention provides a spring-loaded needle free injector for injecting fluid from an injection cartridge that has an injection orifice and a displaceable plunger. Included is an injector body, a main spring for providing injection power for the injector, a ram disposed in the injector body for transferring power from the main spring to the plunger, and a cartridge loading mechanism that retracts the ram for alternatively loading injection fluid into the injection cartridge or for facilitating the loading of a pre-filled injection cartridge.

A needle-free injector for injecting fluid from an injection cartridge that has a longitudinal axis, an injection orifice at a distal end and a displaceable plunger may also be provided in accordance with the invention. The injector typically includes an injector body having a longitudinal axis and a side opening to receive the cartridge from a position laterally offset from the injector body longitudinal axis. A closure mechanism is also included for closing the side opening of the injector body after the cartridge is in position in the injector body to lock the cartridge in the injector body. A ram is disposed in the injector body for transferring power from the system to the plunger, the ram being aligned with the longitudinal axis of the cartridge when the cartridge is in the injector body. A cartridge loading mechanism is normally also included that retracts the ram for alternatively loading injection fluid into the injection cartridge or for facilitating the loading of a pre-filled injection cartridge.

A spring-loaded needle-free injection system is further provided that includes a nozzle having a fluid chamber therein for containing injectable fluid and an injection orifice fluidly coupled with the fluid chamber. Also included is a main spring configured to be compressed during arming of the injection device, the spring-powered injection device being configured to forcibly eject fluid from the fluid chamber out through the injection orifice during decompression of the spring. This system typically also includes a filling adapter that is frangibly attached to the nozzle such that the filling adapter cannot be reattached to the nozzle after being broken away from the nozzle. Thus, the system is configured to prevent delivery of an injection from the injection orifice into an injection site until the filling adapter is broken away from the nozzle.

### BRIEF DESCRIPTION OF THE DRAWINGS

FIG. 1 is a perspective view of a first embodiment of the present invention after the cartridge has been inserted in the injector but before the cartridge is locked in position.

FIG. 2 is a perspective view corresponding to FIG. 1 except that the cartridge has been locked into position.

FIG. 3 is a perspective view corresponding to FIG. 1 except that the trigger sleeve has not yet been slid forwardly as the injector is pressed against the patient.

FIG. 4 is an end view of the first embodiment.

FIG. 5 is an exploded view of the first embodiment.

FIG. 5A is an enlarged perspective view of the ratchet rings of the first embodiment.

FIG. 6 is a side elevation sectional view of the first embodiment before compression of the main spring begins.

FIG. 6A is a fragmentary side elevation sectional view of the first embodiment after the main spring has been loaded.

FIG. 6B is a fragmentary side elevation sectional view of the first embodiment after the main spring has been loaded, and after the trigger sleeve has slid forward to fire but in the instant before firing takes place.

FIG. 6C is a fragmentary side elevation view of the first embodiment after the injector has been fired (corresponding to FIG. 6).

FIG. 7 is a side elevation sectional view of the first embodiment after the main spring has been compressed but before the nozzle has been filled with injection fluid.

FIG. 8A is a side elevation sectional view of the first embodiment after the main spring has been compressed and after the nozzle has been filled with injection fluid.

FIG. 8B is a side elevation sectional view corresponding to FIG. 8A except that section is 90° offset.

FIG. 9 is a side elevation sectional view of a second embodiment before compression of the main spring.

FIG. 10A is a side elevation sectional view of the second embodiment after compression of the main spring and after the nozzle has been filed with injection fluid.

FIG. 10B is a side elevation sectional view corresponding to FIG. 10B except that the section is 90° offset.

FIG. 11 is a side elevation sectional view of the second embodiment after compression of the main spring but before the nozzle has been filled with injection fluid.

FIG. 12 is a side elevation sectional view of the winder portion at the proximal end of the first embodiment.

FIG. 13 is a fragmentary side elevation sectional view of a third embodiment.

FIG. 14 is a fragmentary perspective view of the plunger with ram portion of the third embodiment showing the frangible member broken to prevent re-use.

FIG. 15 is a fragmentary perspective view of the plunger/ram portion of the third embodiment, prior to the point at which the frangible member is broken.

FIG. 16 is a fragmentary perspective view of the plunger and the ram of the third embodiment showing that when the plunger is withdrawn after the frangible member is broken, the plunger does not follow.

FIG. 17 is a perspective view of the plunger of the third embodiment showing the frangible member intact.

FIG. 18 is an exploded isometric view showing alternate embodiments of a vial adapter and nozzle/filling assembly according to the present description.

FIG. 19 is a sectional view depicting operative engagement of the vial adapter and nozzle/filling assembly of FIG. 18, so as to enable a dose of injectable fluid from an external supply (e.g., a vial) to be loaded into the injection device.

FIG. 20 depicts a non-compliant attempt to fill the nozzle/filling assembly of FIGS. 18 and 19 after detachment of the filling adapter.

FIG. 21 is a partial sectional view depicting a further alternate embodiment of a vial adapter and nozzle/filling assembly according to the present description.

FIG. 22 is an exploded isometric view showing another alternate embodiment of a vial adapter according to the present description.

FIG. 23 depicts the vial adapter of FIG. 22 operatively engaged with an alternate nozzle/filling assembly according to the present description.

FIG. 24 is an exploded view of yet another embodiment to be used with a prefilled cartridge.

FIG. 25 is a side elevation sectional view of the embodiment of FIG. 24 after a new, prefilled cartridge has been mounted in place.

FIG. 26 is a side elevation sectional view of the embodiment of FIG. 24 after the injector has been fired.

FIG. 27 has a side elevation sectional view of the embodiment of FIG. 24 after the ram extension has been withdrawn and the cartridge is ready to be removed and replaced.

### DETAILED DESCRIPTION OF THE PREFERRED EMBODIMENTS

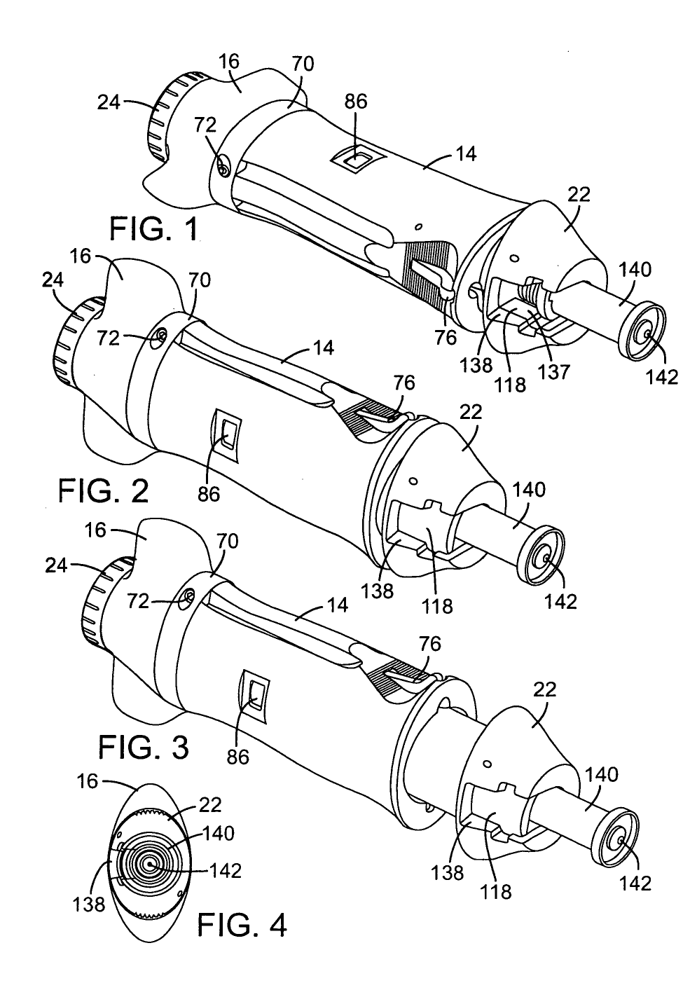

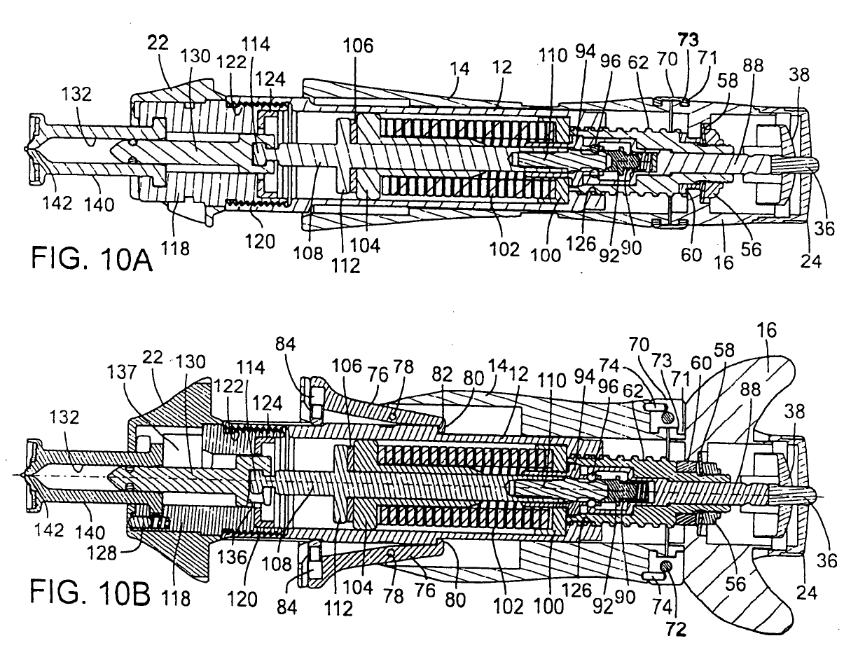

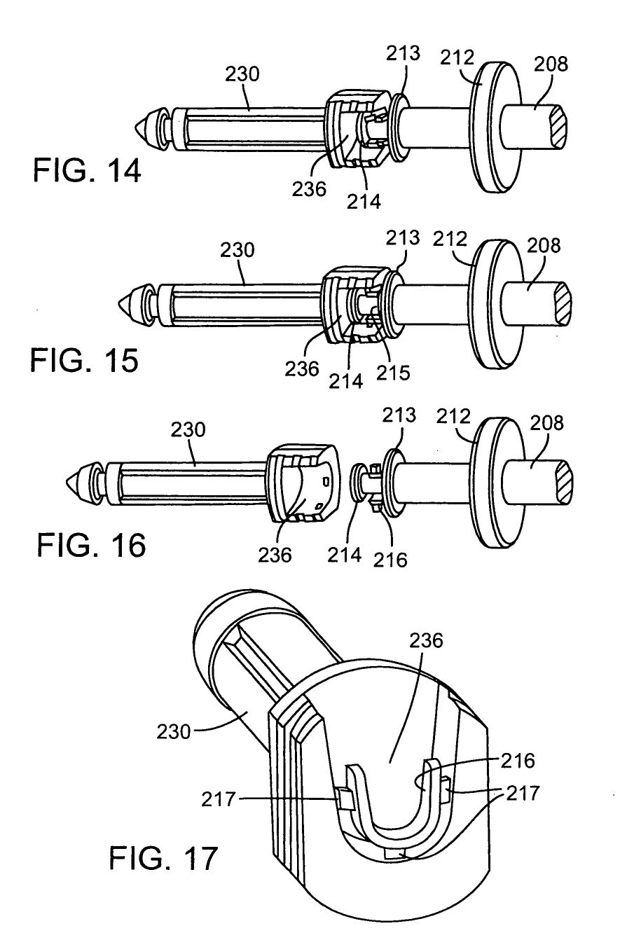

FIGS. 1-27 depict various embodiments of a spring-loaded needle-free injection device. As will be explained in more detail below, the device typically is implemented as a single-use injection system including a fluid cartridge that may be engaged with an injector mechanism such as that depicted in the figures. A fluid chamber within a nozzle/cartridge may be filled with a dose of injectable fluid. Typically, filling is accomplished from an external supply of fluid, which may include a vial adaptor that allows the external supply to be selectively coupled to the nozzle-cartridge filling assembly. After filling, the external supply of fluid is decoupled from the nozzle/cartridge filling assembly by simply removing the external supply and vial adaptor from engagement with the nozzle/cartridge assembly.

### Embodiment of FIGS. 1-8 and 12

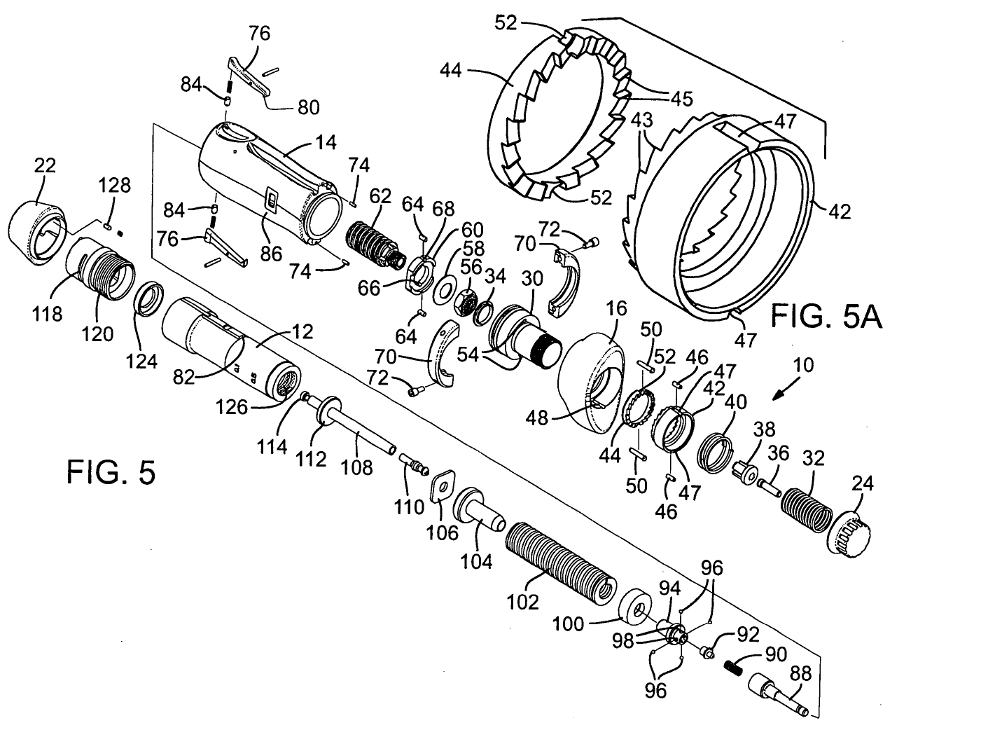

Before describing the operation of the depicted system, the various parts and their relationship to one another will first be described. A first embodiment of the injector system is depicted at 10 in FIGS. 1-8 and 12. For an identification and description of the various parts, reference should first be made to FIGS. 5 and 6-8. The basic components of injector 10 are a main body 12 (see FIG. 5), a trigger sleeve 14, a winder 16, a cartridge 20 and a cartridge lock 22. Trigger sleeve 14 is designed to slideably fit over main body 12. Winder 16 is rotatably mounted to trigger sleeve 14 such that a spring may be compressed to provide power for the injection. Winder 16 is located at a proximal end of injector 10, which is opposite the end to which cartridge 20 is mounted.

Beginning at the proximal end of injector 10, a dosage knob 24 is included. Dosage knob 24 includes fine, left-handed threads 26 which engage complementing fine threads 28 in a dosage drum 30. A dosage spring 32 is positioned within dosage knob 24 and dosage drum 30 and extends between the proximal end of the dosage knob and a dosage spring seat 34. Positioned within dosage spring 32 is a slide bushing extension 36 and a slide bushing extension seat 38. Slide bushing extension 36 and slide bushing seat 38 mount to and extend the length of a slide bushing 88, which will be described in more detail below.

Positioned around dosage spring 32 within the proximal end of injector 10 is an enlarged ratchet spring 40 which is designed to bias a second ratchet ring 42 toward a first ratchet ring 44. The first and second ratchet rings each include a plurality of teeth 45 and 43, respectively (see FIG. 5A), that are designed to permit relative rotation in one direction but not another. The teeth can be seen to be tilted to one side or sloped to facilitate this sliding in one direction and engagement in the other direction. Therefore, when winder 16 causes second ratchet ring 42 to rotate in a clockwise direction, the spring bias provided by ratchet ring 40 causes teeth 45 and 43 to engage and thereby rotate first ratchet ring 44 in that first direction. However, when winder 16 is rotated in a counterclockwise direction, teeth 45 and 43 of first and second ratchet rings 44 and 42 are permitted to slide over one another

A pair of small winder pins 46 are positioned within second ratchet ring slots 47 and winder slots 48 so that when the two ratchet rings are positioned within winder 16, relative rotation is not permitted between the second ratchet ring and the winder.

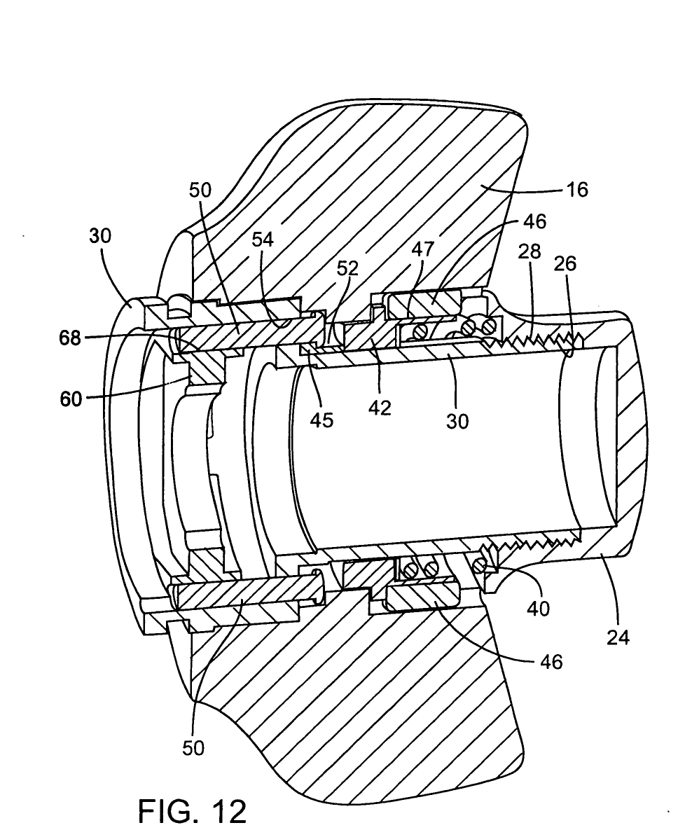

As seen best in FIGS. 5 and 12, a pair of long pins 50, each having a head at its proximal end, are positioned within notches 52 in the outer diameter of first ratchet ring 44, and extend through a pair of diametrically opposed holes 54 in dosage drum 30. Long pins 50 extend in a distal direction past dosage spring seat 34, nut 56, washer 58, and engage notches 68 in the outer diameter of a torque nut 60. A pair of dosage screw pins 64 extend between notches 66 in the inner diameter of torque nut 60 and a dosage screw 62. Thus, dosage screw pins 64 prevent relative rotation between torque nut 60 and dosage screw 62. Because long pins 50 prevent relative rotation between first ratchet ring 44, dosage drum 30 and torque nut 60, relative rotation is not permitted between first ratchet ring 44 and dosage screw 62, for reasons that will become apparent as this description continues.

A pair of so-called clam shell halves 70 are mounted between dosage drum 30 and trigger sleeve 14 to prevent axial displacement between these two components, but permit relative rotation therebetween. Clam shell halves 70 are held together by a pair of clam shell screws 72. Clam shell halves 70 are engaged with trigger sleeve 14 by a pair of clam shell pins 74. The only engagement between clam shell halves 70 and dosage drum 30 is the engagement of a proximal leg 71 of the substantially U-shaped clam shell halves. That is, proximal leg 71 engages a complementing slot 73 in dosage drum 30. Thus, again, the dosage drum, winder and associated parts are held in engagement with the trigger sleeve, but relative rotation is permitted between them so that the winder can be rotated to compress a main spring 102, as will be understood as this description continues.

Continuing in a distal direction, a pair of trigger locks 76 are pivotally mounted to trigger sleeve 14 by trigger lock pivot points 78. Trigger locks 76 each include radially-extending trigger lock legs 80 that engage a ledge or notch 82 in main body 12. Each of the trigger locks 76 includes a trigger lock spring 84 that pushes the distal end of the trigger locks outwardly, thereby causing trigger lock legs 80 to engage notch 82 until the trigger locks are depressed against the outward bias of the trigger lock springs. In most applications only a single trigger lock will be included even though two such trigger locks are included in the depicted embodiment.

A trigger sleeve window 86 is provided in the side of trigger sleeve 14 so that a visual indicator can be provided to ensure the proper positioning of the components prior to firing. Window 86 can also be used to provide a read-out of the dosage that is being injected.

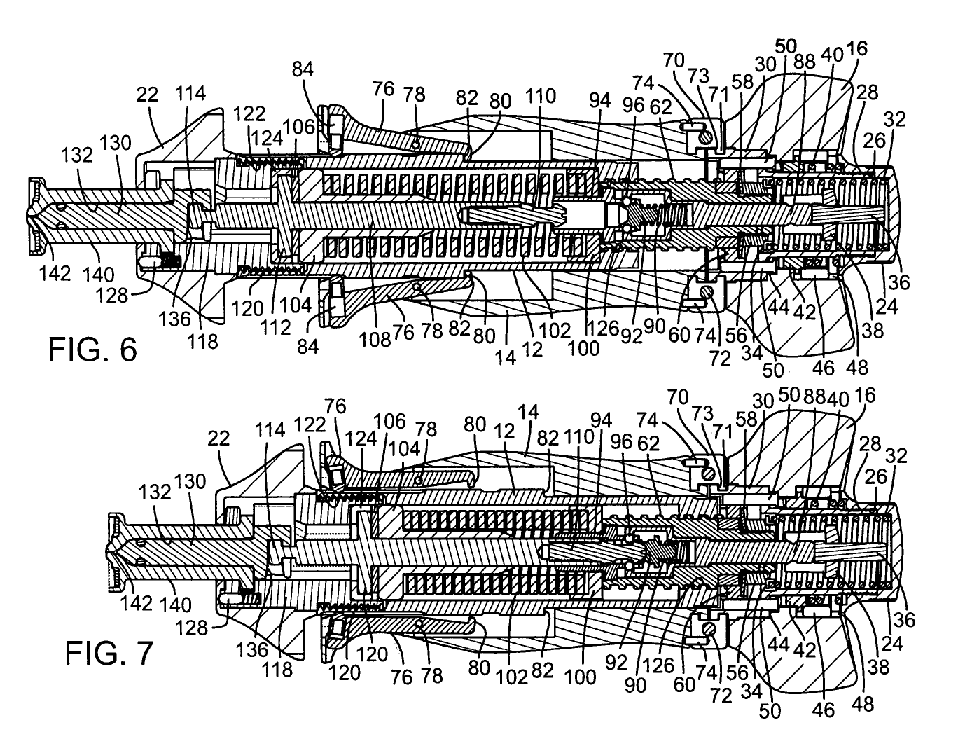

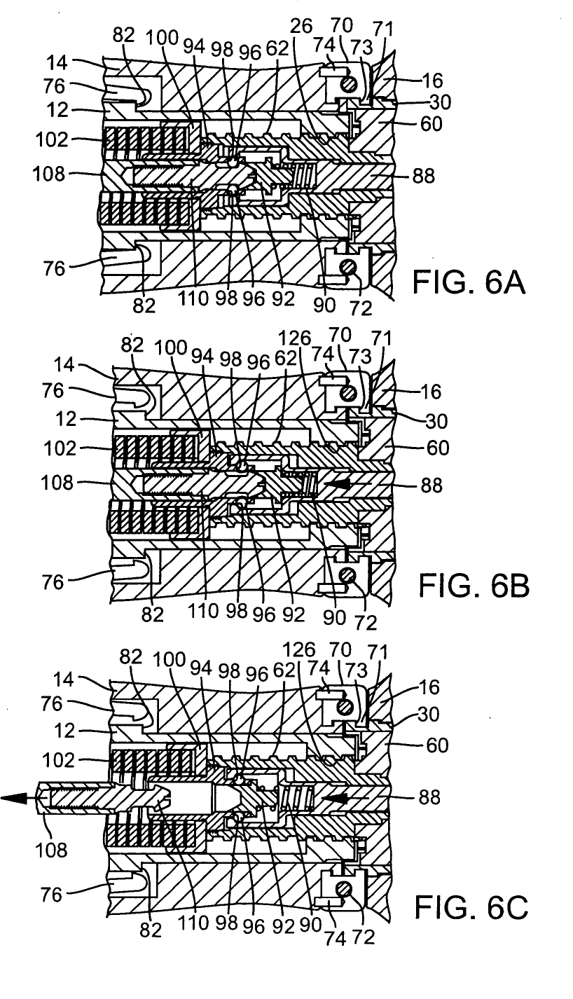

Referring again to the exploded view of FIG. 5 and the assembled view of FIGS. 6-8, slide bushing 88 can be seen to extend through trigger sleeve 14 and within winder 16 to contact the distal end of slide bushing extension 36. In some embodiments there may be a single slide bushing rather than the two-part slide bushing/slide bushing extension shown in injector 10.

A trigger spring 90 can be seen to the positioned within slide bushing 88. Trigger spring 90 is seated in a trigger spring seat 92 which in turn is positioned within a firing sleeve 94. Four hardened steel balls 96 are initially positioned within four ball seats 98 in firing sleeve 94 for purposes that will become apparent as this description continues.

An upper spring seat 100 provides a proximal seat for main spring 102, which provides injection power for injector 10. A main spring seat 104 provides a distal seat for main spring 102. A substantially square washer 106 is shown to be positioned between main spring seat 104 and a ram 108. As shown, main spring seat 104 includes a central opening through which ram 108 extends. A ram bolt 110 extends out of the proximal end of ram 108 to provide a hardened surface for the proximal end of ram 108. Ram 108 includes a ram seat 112 and, at its distal end, a head 114 which is defined by a notch in the ram. The configuration of head 114 is designed to facilitate engagement of cartridge 20.

At the distal end of injector 10 is a cartridge lock 22, which is mounted to main body 12 by a cartridge holder 118. Specifically, external threads 120 in cartridge holder 118 engage with complementing internal threads 122 in main body 12 in order to properly engage the cartridge holder to the main body. A detent pin 128 and a small spring are provided to cause cartridge lock 22 to click into its locked position.

Cartridge 20 can be seen to include a plunger 130 positioned within a chamber 132 in a nozzle 140. The distal end of nozzle 140 includes an injection orifice 142. Plunger 130 includes a substantially U-shaped proximal end 136, which is designed to engage head 114 in the distal end of ram 108. This provides a solid mount that will convey forces conveyed between the ram and the plunger and yet permits easy engagement and disengagement.

Cartridge lock 22 includes a cartridge lock opening 138 (FIGS. 1-4) so that the cartridge can be moved into position from one side and the U-shaped proximal end 136 of plunger 130 may be engaged with head 114 in ram 108. A cartridge holder opening 137 is also provided so that in the cartridge insertion condition, cartridge lock opening 138 is in alignment with the cartridge holder opening. As shown in FIGS. 1 and 2, cartridge lock 22 is then rotated 90° with respect to the rest of injector 10 so that cartridge lock opening 138 and cartridge holder opening 137 are no longer in alignment. This effectively locks cartridge 20 in place in injector 10 for firing.

Nozzle 140 may be loaded with injection fluid by the system described in my application Ser. No. 10/976,342, or any conventional system. Once nozzle 140 is loaded, the nozzle and its injection orifice 142 may be placed against the patient for injection.

While the depicted embodiment is a spring-loaded embodiment, it should be understood that it is also possible to use a gas-powered injector (not shown) in connection with the depicted described system for loading a cartridge from the side. Gas-powered systems are included in U.S. Pat. Nos. 6,096,002, 6,607,510, 6,645,170, and 6,689,093, which are incorporated herein by reference.

FIG. 6 depicts injector 10 in its initial position prior to filling of the nozzle 140 and prior to the point at which main spring 102 is loaded. This is also shown in FIG. 6C. In this position it can be seen that both dosage spring 32 and main spring 102 are in their relaxed positions. Legs 80 of trigger locks 76 are in engagement with notch 82 in main body 12. If the patient now wishes to perform an injection, the patient holds trigger sleeve 14 with one hand, normally the left, and turns winder 16 in a clockwise direction. The ratchet mechanism eases the winding process because winder 16 can merely be turned repeatedly one direction and then the other rather than having to rotate the winder entirely around. In certain applications this may be an easier operation than the complete rotation, particularly for clients who may have decreased motor skills.

As winder 16 is rotated in the clockwise direction, the winder carries second ratchet ring 42. Ratchet spring 40 holds teeth 43 of second ratchet ring 42 against teeth 45 of first ratchet ring 44. This causes first ratchet ring 44 to rotate and along with it so rotates dosage drum 30, torque nut 60 and dosage screw 62. When winder 16 is ratcheted back in a counter clockwise direction, the teeth 45 and 43 of first and second ratchet rings 44 and 42, respectively, slip across each other without causing a reverse rotation of first ratchet ring 44, the dosage drum 30, torque nut 60 or dosage screw 62. As a result of this repeated back and forth rotation of winder 16, dosage screw 62 is turned down into the injector, exerting a forward or downward force on main spring seat 104 and main spring 102 positioned therebelow. This compresses main spring 102 for the injection operation. As the compression of main spring 102 is completed, trigger spring seat 92, firing sleeve 94 and balls 96 move from the position shown in FIGS. 6 and 6C to the position shown in FIGS. 7 and 6A where the balls are positioned immediately below the head of ram bolt 110.

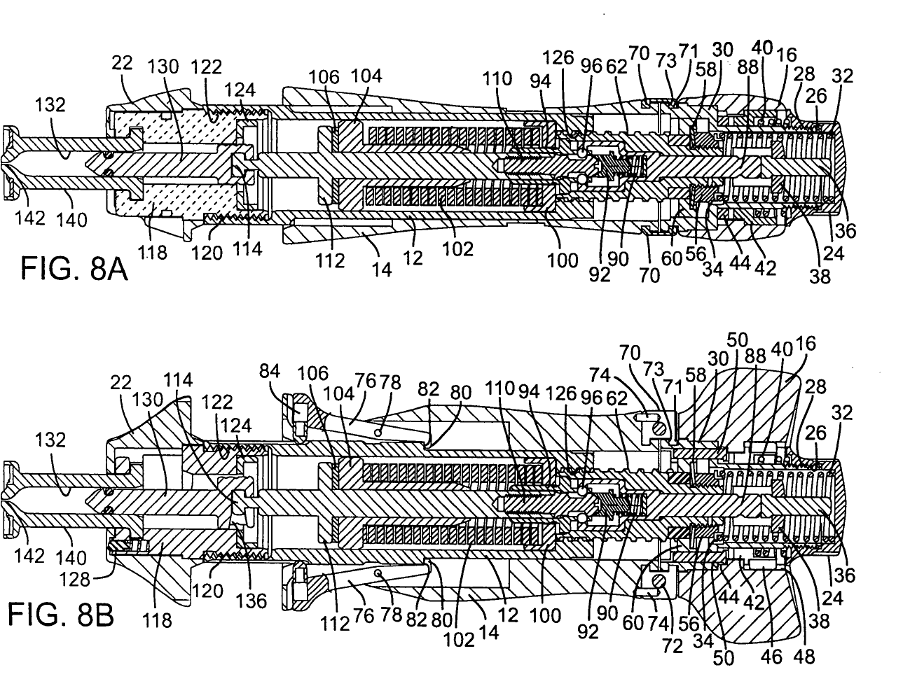

At this point, injector 10 is ready to be loaded with medication, vaccine or other medicinal fluid. In order to retract the plunger and thereby draw fluid from a vial, injector 10 is held in an upright position with the vial at the top. Dosage knob 24 is then rotated in a counter clockwise direction, thereby drawing back slide bushing extension 36, slide bushing extension seat 38, slide bushing 88, firing sleeve 94, ram 108 and plunger 130. This draws fluid into chamber 132, thus preparing injector 10 for injection. This so-called ratchet-ready position is depicted in FIGS. 8A and 8B.

Injector 10 cannot be fired until trigger locks 76 are both depressed, or in the event only one trigger lock is included, the injector cannot be fired until that single trigger lock is depressed. This provides a safety in order to prevent inadvertent firing. To fire injector 10 and inject fluid into the patient, trigger locks 76 are depressed, thereby releasing the engagement between trigger lock legs 80 and notch 82 in main body 12. This is done after orifice 142 of nozzle 140 is pressed against the skin of the patient receiving the injection. Thus, with the trigger locks depressed, injector 10 is pressed against the patient, causing trigger sleeve 14 to slide in a forward direction toward the patient to the position shown in FIG. 7. This causes balls 96 to shift outwardly to the position shown in FIG. 6B. This FIG. 6B shows a disposition of parts that will be only momentary, that is, immediately after balls 96 clear the head of ram bolt 110. Ram 108 will quickly shoot forward, causing plunger 130 to drive fluid in chamber 132 out of orifice 142 and through the skin of the patient.

After the injection process is completed, trigger sleeve 14 is slid back to its original position by spring 32 so that trigger lock legs 80 engage notch 82 of main body, and cartridge lock 22 is rotated to permit sideways removal of nozzle 140. When injector 10 is to be reused, another nozzle is loaded in place and the process is repeated.

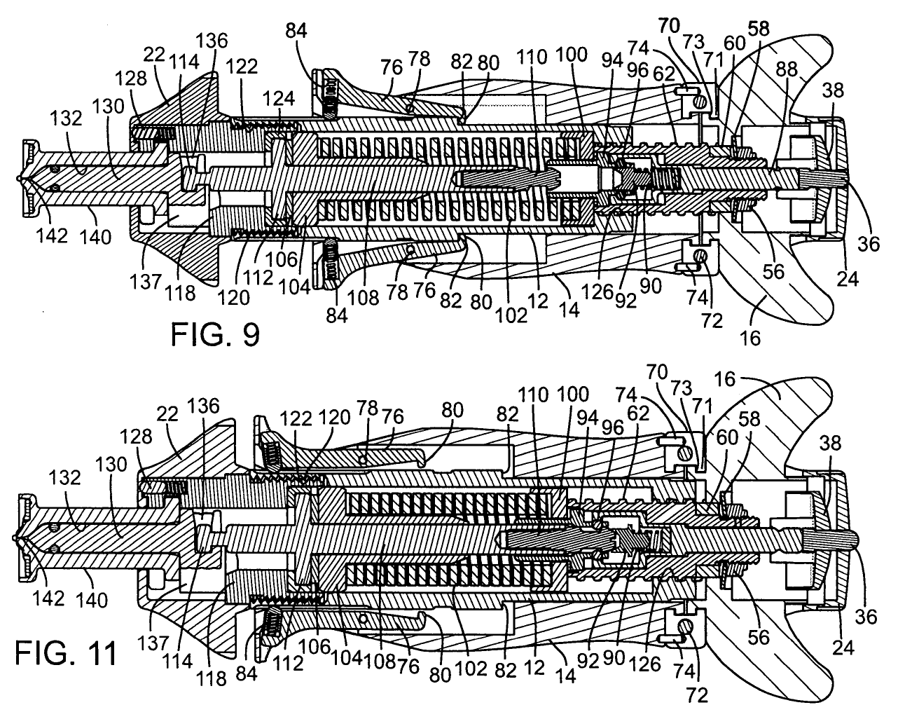

### Embodiment of FIGS. 9-11

The injector of FIGS. 9-11 is identical to the injector of FIGS. 1-8 except that the ratchet mechanism associated with winder 16 has been deleted. Therefore, it is necessary to rotate the winder repeatedly around with respect to trigger sleeve 14 in order to cause main spring 102 to be compressed for firing. It can be seen that the first and second ratchet rings and other associated parts have been deleted from this embodiment 210. Because the other features are typically identical to those described in connection with injector 10, the description of the construction and operation of those other parts will not be repeated. The numbering of the parts has been maintained the same as the embodiment of FIGS. 1-8 and 12 because those parts are typically identical in this second embodiment.

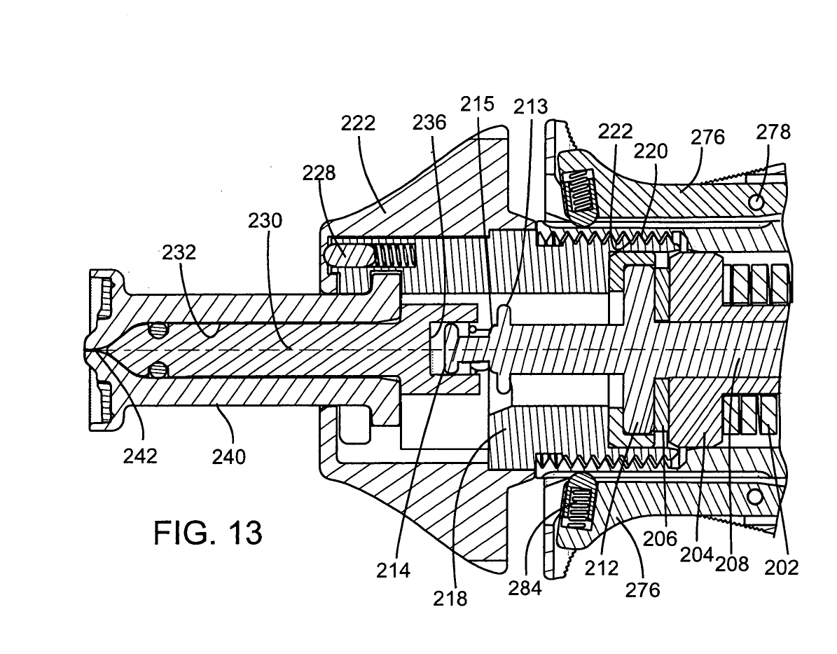

### Embodiment of FIGS. 13-17

FIGS. 13-17 show a slightly different version of the nozzle and ram assemblies. As noted above, it is desirable that once a cartridge or nozzle has been used, that it be disabled so that it cannot be reused. This is desirable to prevent cross-contamination between patients. In order to provide that capability in the disclosed injector 10, the nozzle and ram assemblies may be modified as shown in FIGS. 13-17.

Numbers corresponding to FIGS. 1-8 and 12 are shown, except for similar parts, 100 has been added. Therefore, the nozzle has been identified at 240, the plunger at 230, the nozzle U-shaped proximal end at 236, and the ram at 208. A frangible member 216, shown best in FIG. 17, is mounted to U-shaped proximal end 236 by frangible or breakable tabs 217 for purposes that will become apparent as this description continues.

Instead of notched head 114 in ram 108 of injector 10, the embodiment of FIGS. 1-8 includes two flanges in addition to the flange that forms ram seat 212. As shown, a small distal flange is indicated at 214, and a middle flange is shown at 213. As shown best in FIG. 13, middle flange 213 is disposed proximally of distal flange 214 and includes a shoulder 215 on its distal side.

In operation, nozzle 240 is filled in the same manner as described above with respect to nozzle 140. Ram 208 is drawn back so that distal flange 214 contacts frangible member 216. Because the loading force is so small, perhaps as low as ten pounds or even five pounds or less, frangible member 216 will not break as plunger 230 is pulled back to draw injection fluid into chamber 232.

When the injection force is applied via ram 208, shoulder 215 of middle flange 213 drives through frangible member 216 before distal flange 214 contacts U-shaped proximal end 236 of plunger 230. Shoulder 215 and middle flange 213 close off enough of U-shaped proximal end 236 to prevent fragments of frangible member 216 and tabs 217 from falling out and potentially causing jamming of the various components.

After firing, nozzle 240 is removed from the injector as in the previously-described embodiments. A new nozzle, with an intact frangible member 216, is installed for the next injection. This prevents cross-contamination between patients. If, rather than replacing nozzle 240, the user attempts to reuse and reload the nozzle, the absence of frangible member 216 will cause distal flange 214 to merely pull out of U-shaped proximal end 236 as shown in FIG. 16. This will prevent plunger 230 from being drawn back in chamber 232, and a fluid-loading suction will not be created. Thus, this embodiment of the present invention provides a simple yet effective way to prevent cross-contamination and is a reason this is a preferred embodiment.

As with the first embodiment discussed above, it should be understood that it is possible to use this embodiment of FIGS. 13-17 with a gas-powered injector such as those systems described in U.S. Pat. Nos. 6,096,002, 6,607,510, 6,645,170, and 6,689,093, which are incorporated hereby reference.

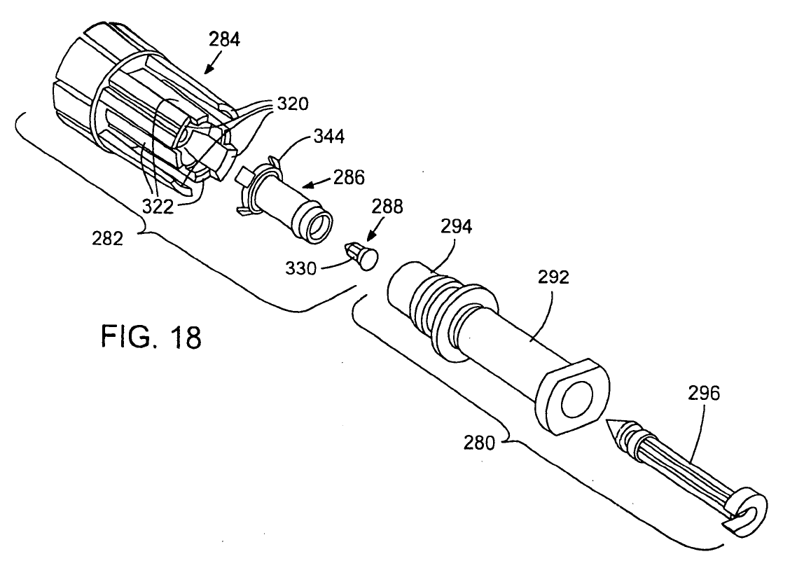

### Embodiment of FIGS. 18-20

Another manner in which cross-contamination can be prevented is to use one of the loading vial adaptor systems described in the parent application. To avoid confusion, the numbering has been retained from the parent application. FIGS. 18-20 depict an embodiment of a nozzle/filling assembly 280 and vial adapter 282. Vial adapter 282 typically includes a main body 284, an inner valve sleeve 286 and a plug 288. Vial adapter 282 typically is attached to and carried on a multiple-dose container (e.g., vial 290) of injectable fluid. Nozzle/filling assembly 280 may include a nozzle 292, a filling adapter 294 secured to the front end of the nozzle, and a piston 296 slidably disposed within a fluid chamber 298 of the nozzle.

Nozzle/filling assembly 280 typically is provided to the end user in a ready-to-fill state. In this state, the nozzle/filling assembly may be operatively engaged with vial adapter 282 to perform the filling operation, in which a dose of injectable fluid is drawn from vial 290 through injection orifice 300 and into fluid chamber 298 of nozzle 292. To allow the injection to go forward, filling adapter 294 is broken away from nozzle 292. Filling adapter 294 is specially configured to operatively engage with vial adapter 282 to perform the filling operation. Typically, the system is configured so that filling cannot occur after filling adapter 294 is broken away. Thus, a single simple step permits the injection to go forward, while simultaneously disabling the ability to refill nozzle 292.

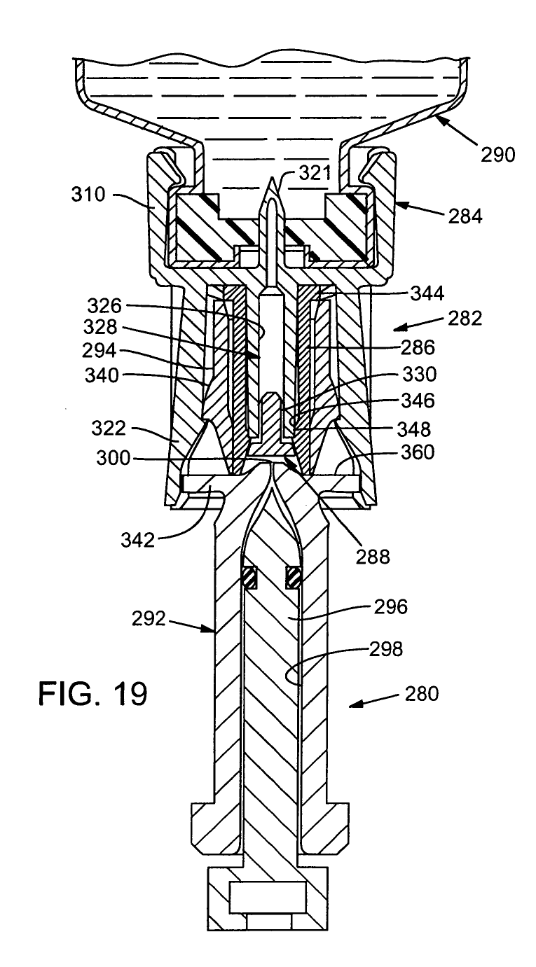

Main body 284 of vial adapter 282 includes a vial gripping section 310 (see FIG. 19) adapted to grip a vial of injectable fluid (e.g., vial 290), and several fingers extending axially away from the gripping section. The extending structures may include relatively rigid fingers 320 and relatively flexible fingers 322 (see FIG. 18). In the depicted embodiment, there are four rigid fingers, with a flexible finger disposed between each rigid finger, for a total of eight fingers, though it should be appreciated that different numbers of fingers may be employed in various configurations.

Vial adapter 282 includes a piercing member or spike 321 configured to pierce a sealed opening of vial 290. Openings are provided on piercing member 321 to enable injectable liquid from vial 290 to flow into a central channel 326 defined within a cylindrical member 328 extending away from gripping section 310 between fingers 320 and 322. Plug 288 is fitted snugly into the distal end of cylindrical member 328. As indicated in FIGS. 18 and 19, plug 288 includes channels 330 configured to permit fluid to be drawn out of central channel 326 and into the area around injection orifice 300 of nozzle 292. As will be explained in more detail, inner valve sleeve 286 may be axially movable between a position in which it seals off channels 330, and an unsealed position, in which liquid is permitted to pass out through the channels to injection orifice 300.

Referring specifically to FIG. 19, to fill the device, nozzle/filling assembly 280 is first inserted into and received within vial adapter 282. Prior to this, nozzle/filling assembly 280 may first be secured within an injector device or other mechanism. As nozzle/filling assembly 280 is inserted into vial adapter 282, a ramped portion 340 on the outer diameter of filling adapter 294 bears against flexible fingers 322, urging them outward. Flexible fingers 322 are urged far enough outward by filling adapter 294 so that the flexible fingers are pushed beyond the outer edges of a flanged portion 342 of nozzle 292, thereby allowing the nozzle/filling assembly to be inserted further into vial adapter 282.

Inserting nozzle/filling assembly 280 into vial adapter 282 also causes a forward end of nozzle 292 to push against the distal end of inner valve sleeve 286. Prior to contact with nozzle 292, inner valve sleeve 286 is biased axially away the vial-gripping portion of vial adapter 282 by resilient feet 344 provided on the proximal end of inner valve sleeve 286. In this initial position, an annular protruded area 346 on the inner diameter of inner valve sleeve 286 seals channels 330 formed in plug 288, thereby preventing liquid from passing out of central channel 326.

The insertion of nozzle/filling assembly 280 into vial adapter 282 pushes the inner valve sleeve 286 axially toward vial 290, compressing feet 344 and moving the sleeve so that the annular protruded area 346 does not seal channels 330 (FIG. 19). Piston 296 may then be drawn back to draw a dose of injectable liquid into fluid chamber 298 of nozzle 292. To create suction, the outer diameter of inner valve sleeve 286 may also be provided with an annular protruded area 348 to seal against the inner diameter of filling adapter 294.

After piston 296 has been withdrawn to draw in a dose of injectable fluid, filling adapter 294 may be broken away from nozzle 292. Typically, nozzle/filling assembly 280 is manufactured so that there is a frangible or breakable connection 360 between filling adapter 294 and nozzle 292 at the desired breaking point. Typically, after the filling adapter is broken away, it cannot be reattached to the nozzle by the user.

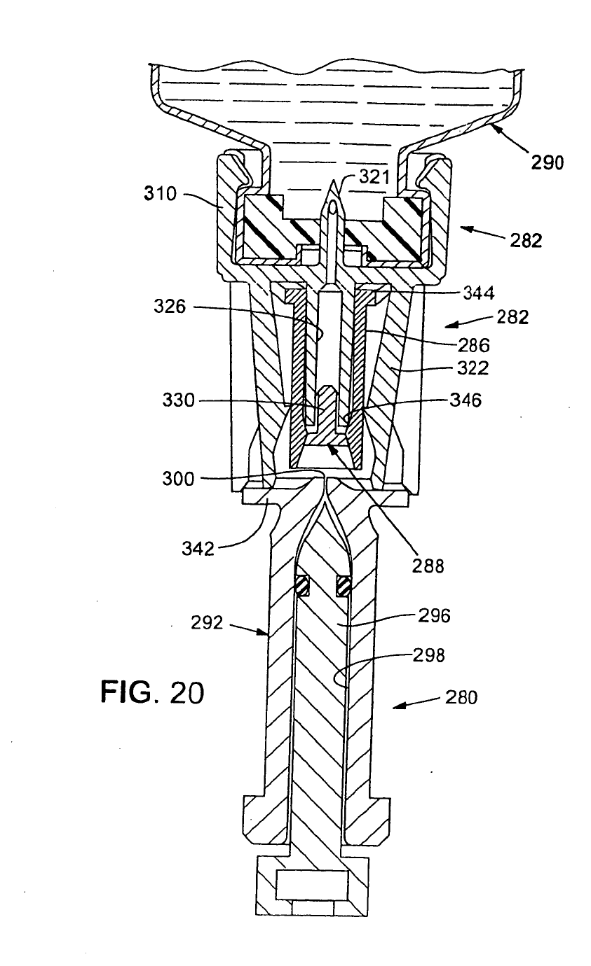

Referring now to FIG. 20, it will be appreciated that the described exemplary system prevents filling after the filling adapter has been broken away. Specifically, the figure depicts a non-compliant attempt to engage vial adapter 282 with nozzle 292 after the filling adapter has been broken away from the front of nozzle 292 (e.g., after an injection has been delivered). As shown, flexible fingers 322 of vial adapter 282 are biased inward so as to block the flanged portion 342 of nozzle 292 surrounding injection orifice 300. Since filling adapter 294 (FIGS. 18 and 19) has been broken away, no structure remains to spread the flexible adapter structure outward away from the blocking position to allow further axial movement of nozzle 292 toward vial adapter 282.

Because the flexible fingers act as a blocking mechanism or outer protective shroud that maintains nozzle 292 spaced apart from the end of inner valve sleeve 286, the respective fluid paths of vial adapter 282 and nozzle 292 are prevented from coming into contact, thereby guarding against contamination. Also, the nozzle is prevented from pushing against the end of inner valve sleeve 286, such that the nozzle cannot push the inner valve sleeve inward to disable the sealing of channels 330 by annular protruded area 346. Furthermore, because filling adapter 294 has been removed, a seal cannot be established to seal an enclosed area between the fluid paths. Accordingly, it should be appreciated that the removal of filling adapter 294 guards against contamination, prevents refilling and otherwise protects against unintended use.

As in the previous examples, the device depicted in FIGS. 18-20 is configured to prevent delivery of an injection until the filling adapter is broken away and the refilling capability disabled. Specifically, the filling adapter may be disposed on the nozzle and sized so that the injection orifice is sufficiently spaced from the injection site so as to prevent an effective injection from occurring.

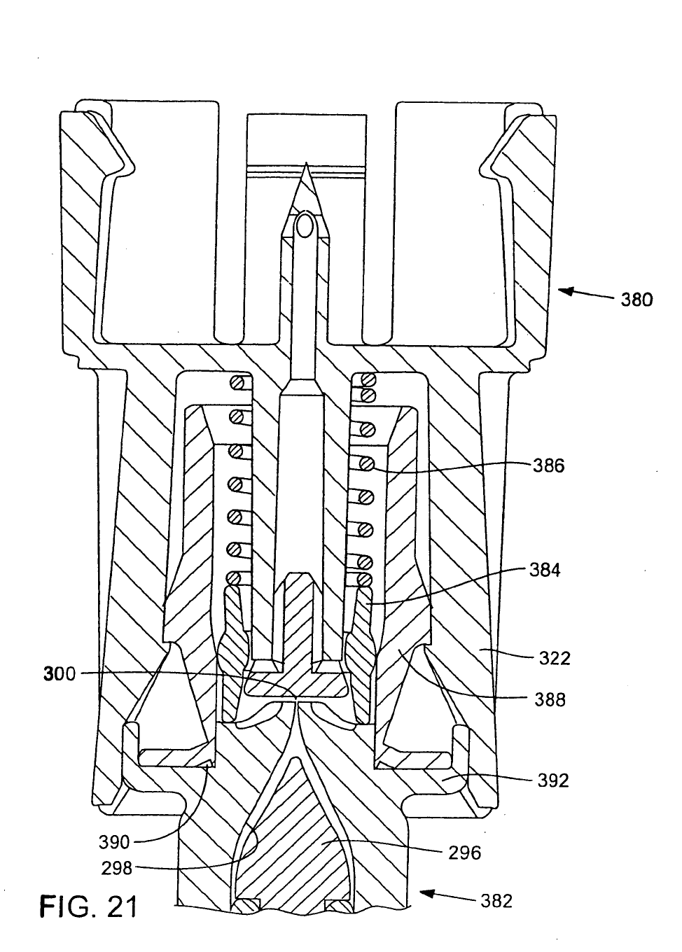

### Embodiment of FIG. 21

FIG. 21 depicts a further alternate embodiment of a vial adapter 380 and nozzle/filling assembly 382 Vial adapter 380 differs from the vial adapter of FIGS. 18-20 in that it includes an alternate inner valve sleeve 384 which is biased into a sealed position by a spring 386. In the sealed position (not shown), the inner diameter of valve sleeve 384 seals channels 330 of plug 288. As in the example of FIGS. 18-20, nozzle/filling assembly 382 includes a filling adapter 388 that spreads flexible fingers 322 apart to enable the components to be positioned axially close enough to one another to defeat the sealing of channels 330 and create suction to allow fluid to be drawn into fluid chamber 298 upon retraction of piston 296. During retraction of piston 296, the outer diameter of valve sleeve 384 seals against the inner diameter of filling adapter 388 to create suction.

Also, nozzle/filling assembly 382 differs from that of FIGS. 18-20 in that frangible connection 390 is in a recessed location relative to injection orifice 300. Specifically, the frangible connection is spaced axially away in a rearward direction (e.g., rearward along the injection axis) from the generally planar area at the forward end of nozzle 392 that is placed onto the injection site during delivery of an injection. This may be desirable in certain applications, to ensure that sharp edges or other irregularities resulting from breakage are prevented from coming into contact with the injection site (e.g., a patient's skin). Also, as indicated, filling adapter 388 may be fabricated as a separate piece, rather than integrally formed with nozzle 392. In the depicted example, the separate filling adapter piece may be ultrasonically welded to nozzle 392 or secured in place with any other desired method.

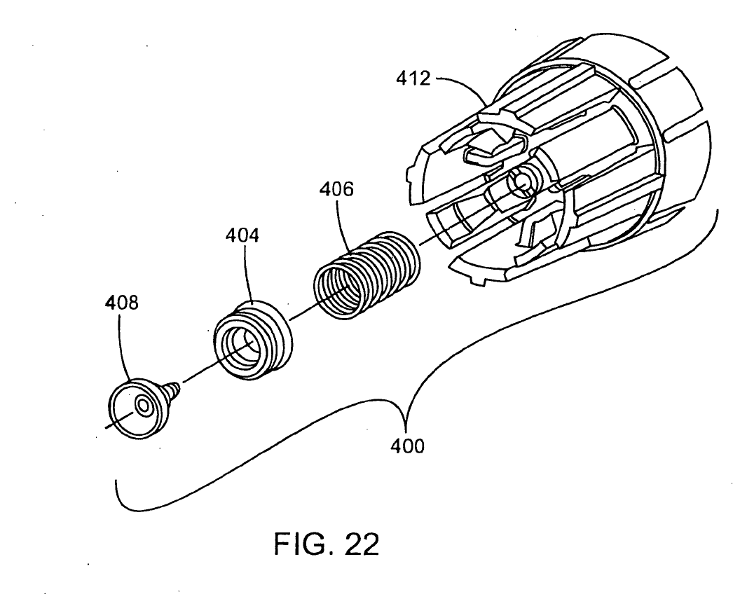

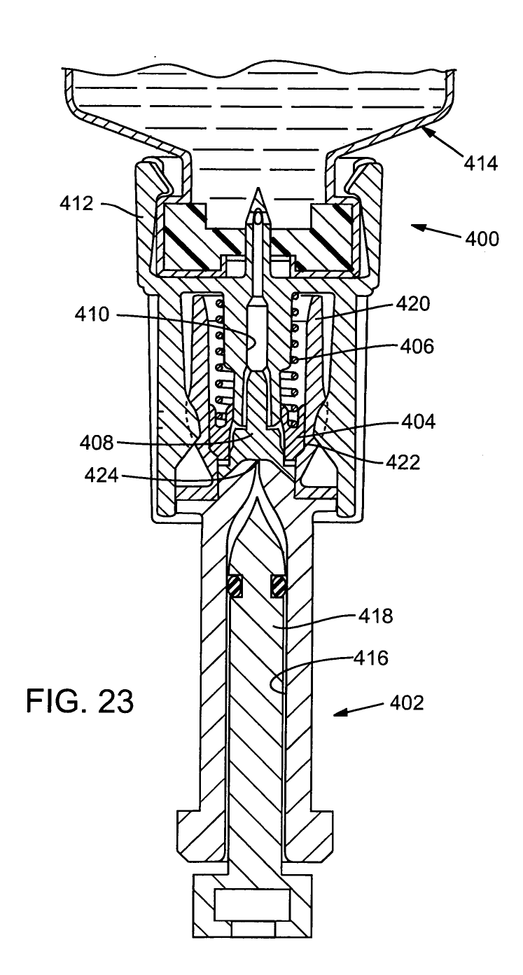

### Embodiment of FIGS. 22 and 23

FIGS. 22 and 23 depict a further alternate embodiment of a vial adapter 400 and nozzle/filling assembly 402 according to the present disclosure. Similar to the example of FIG. 21, vial adapter includes a valve sleeve 404 which is biased (downward in FIG. 23) into a sealed position by a spring 406. A plug 408 is fitted into a cylindrical passage 410 of the vial adapter main body 412. As in the previous exemplary embodiments, plug 408 includes channels through which fluid can flow from vial 414 out through passage 410 and out of the vial adapter (e.g., into variable volume fluid chamber 416 in which plunger 418 is disposed). However, a lower end of sleeve 404 seals these channels until the sleeve is moved out of the sealing position (e.g., moved upward against the spring tension via engagement of the vial adapter with an appropriately shaped filling adapter).

FIG. 23 depicts engagement of nozzle/filling assembly 402 with vial adapter 400. As shown, filling adapter 420 may include on its inner diameter a circumferential ledge 422 sized to bear against a lower portion of valve sleeve 404 when the components are brought together. This urges valve sleeve 404 upward against the force of spring 406, as shown in the figure, such that fluid is now permitted to pass from passage 410, through the channels in plug 408, and into the injection device through injection orifice 424.

It will be appreciated that the nozzle/filling assembly 402 and vial adapter 400 provide similar advantages to the other embodiments discussed herein. In particular, filling adapter is configured so that it is frangibly connected to the nozzle, and must be broken away before an injection can be administered. As in the other embodiments, this breaking of the filling adapter prevents reuse by disabling the ability to refill the device. Specifically, once the filling adapter has been removed, the nozzle is no longer shaped to engage the opening of the vial adapter and actuate the adapter valve seal. Also, the vial adapter has an outer structure, as in previous embodiments, that acts as a protective shroud to protect the fluid pathway and reduce risk of contamination.

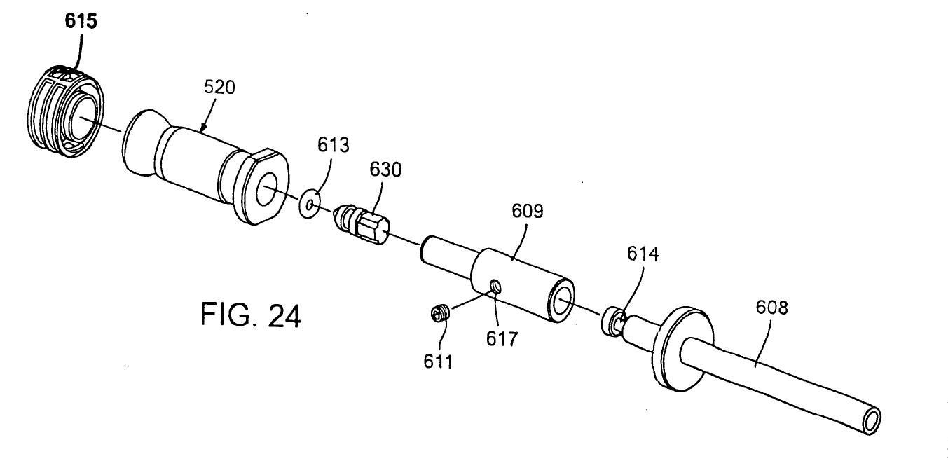

### Embodiment of FIGS. 24-27

FIGS. 24-27 depict an embodiment 510 designed to be used with a prefilled cartridge 520. Injector 510 is virtually identical to injector 10 except that a ram extension 609 is included, and is mounted to ram 608 by a set screw 611. When mounted in place, set screw 611 is threaded into a threaded hole 617 in ram extension 609 so that the set screw extends into a notch 614 in ram 608. Plunger 630 is much shorter than plunger 130, and includes an O-ring 613. A cap 615 is also shown in FIG. 24, and cartridge 520 is slightly modified from cartridge 30 of injector 10. For example, cartridge 520 is typically fabricated of Topas® cyclic olyfin copolymer (COC) from Celanese/Ticona. This material has been found to be relatively inert and therefore normally will not react with typical formulations stored in the cartridge. Other parts that are the same as those previously described and depicted in FIGS. 1-8 and 12 have been numbered by simply adding 500 to the numbers used in injector 10 shown in FIGS. 1-8 and 12. The description of the structure and operation of those parts will not be repeated.

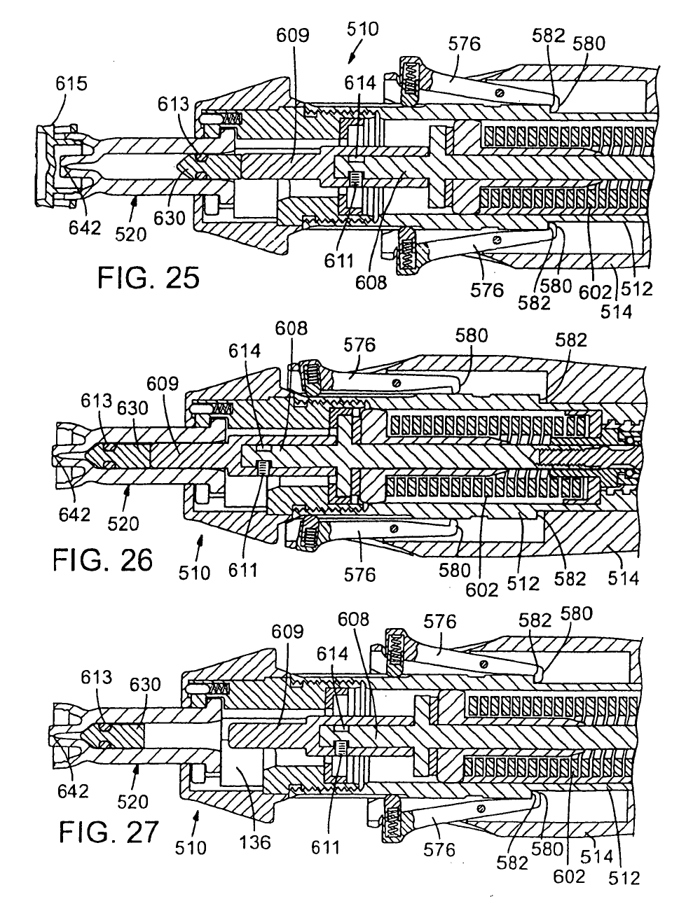

The operation of injector 510 can be understood by making reference to FIGS. 25-27. FIG. 25 shows injector 510 after prefilled cartridge 520 has been positioned in the injector but before firing. Ram extension 609 is shown to be mounted to ram 608 by set screw 611. Plunger 630 is positioned in the proximal end of prefilled cartridge 520, with cap 615 in place. In FIG. 25 main spring 602 is shown to be compressed using a winder (not shown), just like as in injector 10 that has been previously described.

In order to fire injector 510, trigger locks 576 are depressed, disengaging trigger lock legs 580 from notch 582 in main body 512. This permits the operator to slide trigger sleeve 514 forwardly on main body 512 as injector 510 is pressed against the patient. This releases main spring 602 as previously described, driving ram 608, ram extension 609 and plunger 630 forwardly or in a distal direction. This causes fluid to be ejected out orifice 642 and into the patient. This just-fired position of the components is shown in FIG. 26.

To prepare injector 510 for the next injection, the winder (not shown) compresses main spring 602 using the ratcheting operation previously described. Alternatively, the continuous rotation embodiment of the winder mechanism can be substituted. A dosage knob (not shown), like dosage knob 24 of injector 10, is turned, and this retracts ram 608 and ram extension 609 mounted to it. This facilitates the remove and replacement of cartridge 520 through the side of injector 510 as previously described in connection with injector 10. Because the distal end of ram extension 609 is normally perfectly positioned to abut the proximal end of plunger 630, injector 510 is now ready for the next injection (as shown in FIG. 25).

While various embodiments and arrangements of a needle-free injection system and method have been shown and described above, it will be appreciated that numerous other embodiments, arrangements, and modifications are possible and are within the scope of the invention. The foregoing description should be understood to include all novel and non-obvious combinations of elements described herein, and claims may be presented in this or a later application to any novel and non-obvious combination of these elements. The foregoing embodiments are illustrative, and no single feature or element is essential to all possible combinations that may be claimed in this or a later application.

We hereby claim:

1. A needle-free injector for injecting fluid from an injection cartridge that has a longitudinal axis, an injection orifice at a distal end and a displaceable plunger, comprising:
an injector body having a longitudinal axis and a side opening to receive the cartridge from a position laterally offset from the injector body longitudinal axis; and
a closure mechanism for closing the side opening of the injector body after the cartridge is in position in the injector body with the longitudinal axes of the cartridge and the injector body coincident in order to lock the cartridge in place in the injector body.

2. The injector of claim 1 wherein the closure mechanism includes a rotatable cartridge lock that rotates on an axis that is coincident with the injector body longitudinal axis.

3. The injector of claim 2 further comprising a cartridge holder disposed in the injector body at a position that is radially offset from the cartridge lock, the cartridge holder not being rotatably mounted with respect to the injector body, the cartridge holder further including a side opening that is at least partially coincident with the side opening in the injector body when the cartridge lock is unlocked so that the cartridge may be inserted from the side through the side opening in the injector body and the side opening in the cartridge holder, and so that when the cartridge lock is rotated to its locked position, the cartridge is locked in position in the injector.

4. The injector of claim 3 wherein the cartridge holder is disposed radially inwardly of the cartridge lock.

5. The injector of claim 3 where in the side openings of the cartridge holder and the cartridge lock are substantially coincident when the cartridge is unlocked.

6. The injector of claim 1, further comprising a system disposed in the injector body for providing injection power, and a ram disposed in the injector body that is axially aligned with the longitudinal axis of the injector body for transferring power from the system to the plunger.

7. The injector of claim 6 wherein the system includes a main spring for providing injection power.

8. A method for loading an injection cartridge into a needle-free injector, comprising the following steps:
selecting an injection cartridge that has a longitudinal axis;
selecting an injector body that has a longitudinal axis, a side opening and a closure mechanism for selectively closing the side opening;
opening the closure mechanism of the injector body;
moving the injector cartridge laterally from a position that is laterally offset from the longitudinal axis of the injector body through the side opening of the injector body into position in the injector body with the longitudinal axes of the injector body and the cartridge coincident; and
closing the side opening of the injector body to lock the cartridge in place in the injector body.

9. A spring-loaded needle-free injector comprising:
an injector body;
a main spring disposed in the injector body;
a winder that is rotatable in either the first direction or a second directions and which can compress the main spring by rotating in the first direction; and
a ratchet mechanism in the form of a first and a second toothed member with at least one of the members being spring biased toward the other member, the ratchet mechanism being interconnected with the winder so that winding the winder in the first direction causes both toothed members to rotate, but winding the winder in the second direction causes rotation of only the first toothed member.

10. The injector of claim 9 wherein the injector body has a longitudinal axis and the toothed members comprise a pair of toothed rings that are rotatable around the longitudinal axis of the injector body.

11. The injector of claim 9, further comprising a dosage drum that is threaded to the injector such that rotation of the dosage drum in the first direction turns the dosage drum toward the main spring to compress the main spring, the dosage drum being rotationally fixed with respect to one of the toothed members so that when that member rotates, so does the threaded dosage drum.

12. A method for compressing the spring of a spring-loaded needle-free injector comprising:
selecting an injector body having a main spring, a winder that is rotatable in either a first direction or a second direction and which compresses the main spring by rotating in the first direction, and a ratchet mechanism having rotatable first and second members with sloped teeth, with at least one of the members being spring biased toward the other member, the ratchet mechanism being interconnected with the winder so that winding the winder in the first direction causes both toothed members to rotate but winding the winder in the second direction causes rotation of only the first toothed member;
winding the winder in the first direction, causing both of the members to rotate to compress the main spring;
winding the winder in the second direction, causing only one of the members to rotate; and
winding the winder again in the first direction, causing both of the members to rotate to further compress the main spring.

13. A spring-loaded needle-free injector comprising:
a main body having distal and proximal ends and a longitudinal axis;
an injection cartridge that is mounted to the distal end of the main body, the cartridge having a fluid chamber defined therein and an injection orifice at the distal end thereof;
a trigger sleeve that is slidably mounted to the main body;
a trigger lock that can be locked and unlocked and which prevents the trigger from sliding with respect to the main body when the trigger lock is locked; and
a firing mechanism that is actuated by unlocking the trigger lock and then sliding the trigger sleeve toward the distal end of the main body, thereby firing the injector and causing fluid to be injected out of the injection orifice.

14. The injector of claim 13 wherein the trigger lock engages the main body when in its locked position, and by pressing the trigger lock the main body is disengaged from the lock and the trigger sleeve is permitted to slide toward the distal end of the main body.

15. The injector of claim 14 wherein the trigger lock comprises a pivotable member having a leg adjacent one end that engages the main body when the trigger lock is in its locked position, with a second end of the trigger lock protruding from the main body, so that by pressing the second end of the trigger lock the sleeve is disengaged from the main body and is permitted to slide toward the distal end of the main body.

16. A method of injecting fluid from a needle-free injector comprising:
selecting an injector having a main body with distal and proximal ends;
slidably mounting a trigger sleeve to the main body, the trigger sleeve having the capability of actuating a firing mechanism when slid along the main body in a distal direction, the trigger sleeve having a trigger lock that can be locked and unlocked and which prevents the trigger sleeve from sliding with respect to the main body when the trigger lock is locked;
mounting an injection cartridge adjacent the distal end of the main body, the cartridge including a fluid chamber defined therein and an injection orifice at the distal end thereof; and
unlocking the trigger lock and sliding the trigger sleeve toward the distal end of the main body to fire the injector and cause the fluid to be ejected out of the fluid chamber and through the injection orifice.

17. The method of claim 16, further comprising compressing a spring disposed in the injector before unlocking the trigger lock, the spring providing power for the injection.

18. A needle free injector comprising:
an injection cartridge including a plunger and an injection orifice;
an injector body;
a system for providing injection power;
a ram disposed in the injector body for transferring injection power from the system to the plunger;
a frangible member mounting the ram to the plunger such that the ram and plunger can be retracted under one amount of force to fill the cartridge with fluid, and such that when the ram and plunger are driven forward under another amount of force that is greater than the one amount of force, the ram breaks the frangible member but still allows the ram and plunger to drive fluid from the cartridge through the injection orifice, so that in the event someone attempts to retract the ram and plunger to re-fill the cartridge, the ram will retract but the plunger will not because the frangible member has been broken.

19. A needle free injector comprising:
an injector body;
an injection cartridge having distal and proximal ends and having a plunger and defining an injection orifice, the cartridge being removably mounted to the injector body;
a system for providing injection power;
a ram disposed in the injector body for transferring injection power from the system to the plunger, the ram including a distal flange positioned adjacent a proximal end of the plunger, and a proximal flange disposed proximally of the distal flange; and
a frangible member mounted to the plunger such that the frangible member is positioned between the two ram flanges so that retraction of the ram under one amount of force causes the distal flange to contact the frangible member and thereby retract the plunger to fill the cartridge with fluid, and such that when the ram and plunger are driven forward under another amount of force that is greater than the one amount of force, the proximal flange breaks the frangible member but still allows the ram and plunger to drive fluid from the cartridge through the injection orifice, so that in the event someone attempts to retract the ram and plunger to re-fill the cartridge, the ram will retract but the plunger will not because the frangible member has been broken.

20. The injector of claim 19 wherein the one amount of force is no more than about 10 pounds.

21. A method for preventing the re-loading of a cartridge to be used in a needle free injector, comprising:
selecting an injection cartridge having a plunger and an injection orifice; and
selecting an injector body in which the cartridge can be removably mounted and having a system for providing injection power, a ram for transferring injection power from the system to the plunger, and a frangible member mounting the ram to the plunger such that the ram and plunger can be retracted under one amount of force to fill the cartridge with fluid, and when the ram and plunger are driven forward under another amount of force that is greater than the one amount of force, the ram breaks the frangible member but still allows the ram and plunger to drive fluid from the cartridge through the injection orifice, so that in the event someone attempts to retract the ram and plunger to re-fill the cartridge, the ram will retract but the plunger will not because the frangible member has been broken.

22. A spring-loaded needle free injector for injecting fluid from an injection cartridge that has an injection orifice and a displaceable plunger, comprising:
an injector body;
a main spring for providing injection power for the injector;
a ram disposed in the injector body for transferring power from the main spring to the plunger; and
a cartridge loading mechanism that acts to retract the ram for alternatively loading injection fluid into the injection cartridge or for facilitating the loading of a pre-filled injection cartridge.

23. The injector of claim 22 wherein the cartridge loading mechanism is threaded to permit the operator to turn a portion of the injector to retract the ram to load injection fluid or to facilitate the loading of a pre-filled cartridge.

24. The injector of claim 22 wherein the ram is engageable with the plunger so that when the ram and plunger are engaged and the ram is retracted, the plunger is also retracted so that fluid can be drawn into the cartridge.

25. A needle-free injector for injecting fluid from an injection cartridge that has a longitudinal axis, an injection orifice at a distal end and a displaceable plunger, comprising:
an injector body having a longitudinal axis and a side opening to receive the cartridge from a position laterally offset from the injector body longitudinal axis;
a closure mechanism for closing the side opening of the injector body after the cartridge is in position in the injector body to lock the cartridge in the injector body;
a ram disposed in the injector body for transferring power from the system to the plunger, the ram being aligned with the longitudinal axis of the cartridge when the cartridge is in the injector body; and
a cartridge loading mechanism that retracts the ram for alternatively loading injection fluid into the injection cartridge or for facilitating the loading of a pre-filled injection cartridge.

26. The injector of claim 25 wherein the ram is engangeable with the plunger so that when the ram and plunger are engaged and the ram is retracted, the plunger is also retracted so that fluid can be drawn into the cartridge.

27. A needle-free injection system, comprising:
a spring-powered injection device, including a nozzle having a fluid chamber therein for containing injectable fluid and an injection orifice fluidly coupled with the fluid chamber, the injection device further including a spring configured to be compressed during arming of the injection device, the spring-powered injection device being configured to forcibly eject fluid from the fluid chamber out through the injection orifice during decompression of the spring; and
a filling adapter that is frangibly attached to the nozzle such that the filling adapter cannot be reattached to the nozzle after being broken away from the nozzle, and wherein the needle free-injection system is configured to prevent delivery of an injection from the injection orifice into an injection site until the filling adapter is broken away from the nozzle.

28. The system of claim 27, wherein the needle-free injection system is configured to prevent delivery of an injection into an injection site until the ability of the filling adapter to enable filling of the injection device has been disabled.

29. The system of claim 27, wherein the filling adaptor includes a spring-loaded seal for sealing the injection orifice, the seal being able to be opened when the adaptor is mounted to a vial so that injection fluid is able to flow from the vial, through the orifice and into the fluid chamber.
